(19) United States
(12) Patent Application Publication
(10) Pub. No.: US 2009/0270806 A1
(43) Pub. Date: Oct. 29, 2009

(54) Devices and Methods for Controlled-Depth Injection

(75) Inventors: Patrick Macaulay, Windsor, CA (US); Asha Nayak, Sunnyvale, CA (US); Dustin Thompson, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/108,961

(22) Filed: Apr. 24, 2008

(51) Int. Cl. A61M 5/46

(52) U.S. Cl. 604/117

(57) ABSTRACT

Devices and methods for limiting the depth to which a penetrator is advanced into an organ or mass of tissue. The device generally comprises a first member and a second member. The penetrator is attached to and extends from a second member. The first member has a penetrator shroud and a hollow bore extending therethrough. The second member is engageable with the first member such that a distal portion of the penetrator extends through the penetrator shroud. The distance to which the penetrator protrudes out of and beyond the distal end of the penetrator shroud is adjustable in accordance with the desired depth of penetration. The penetrator may then be advanced into the organ or tissue mass until the distal end of the shroud abuts against the organ or tissue mass, thereby stopping further advancement of the penetrator. The penetrator may have one or more lumen(s) for aspirating or infusing substances.

### FIELD OF THE INVENTION

The present invention relates generally to medical devices and methods, and more particularly to devices and methods for controlling the depth at which diagnostic or therapeutic substance(s) is/are injected into a tissue mass or organ of a human or animal subject.

### BACKGROUND

In medicine and surgery, there are numerous occasions wherein it is desirable to limit the depth to which a needle or other elongate penetrator penetrates into an organ or tissue mass. In this regard, various devices have been used to limit the depth to which needles and other devices penetrate. For example, U.S. Pat. No. 5,141,496 describes a syringe guide with adjustment of the depth to which the needle penetrates. One end of the syringe guide has a sliding base which is adjustable by means of a screw and the other end includes a spring-loaded sliding portion that is affixed to the syringe and propels the needle to a predetermined depth of injection.

U.S. Pat. No. 5,250,026 (Ehrlich et al.) describes an implant injector that has an adjustable insertion depth feature. The insertion depth adjusted by moving the nose of the injector relative to the tip of the cannula that extends past the nose. In addition to adjusting the insertion depth, the cannula or needle, may also be rotated to a plurality of positions relative to the injector handle. A spring loaded plunger, when released by a release button, will push the implant out the end of the cannula as the operator withdraws the cannula from the animal. The release button is designed as a safety trigger to avoid premature activation of the plunger during insertion of the needle. Needles, or cannulas of various diameters and lengths, may be interchanged in the injector. Also, the spring loaded plunger for expelling the implant may be removed allowing the operator to replace the plunger with a different diameter and length plunger, if desired, to match different size cannulas.

U.S. Pat. No. 5,102,393 (Sarnoff et al.) describes an autoinjector that has an intramuscular injection mode and a subcutaneous injection mode. An injection mode converting structure is useable to convert the device back and forth between a subcutaneous mode wherein the needle is allows to advance to a first depth that does not extend substantially beyond subcutaneous tissue at the injection site and an intramuscular mode wherein the needle is allowed to advance to a second depth that is within muscle that underlies the subcutaneous tissue.

U.S. Pat. No. 3,538,916 describes an injection pistol for intramuscular implantation of encapsulated liquid or solid chemical material. The depth of injection of the needle is controlled by an injection depth gauge mounted on the injection needle. A shaft having a slidable plunger integral therewith is mounted on the frame and is utilized to eject the material from the needle after the needle has been advanced into the muscle. The travel of the plunger within the injection needle is limited by a threadedly adjustable depth stop mounted on the end of the shaft opposite the plunger.

U.S. Pat. No. 4,270,537 (Romaine) describes a hypodermic syringe and automatic needle insertion device wherein the syringe is biased against a trigger when the needle is in the retracted position. Upon release of the trigger, the syringe and needle are driven forward extending the needle into the underlying tissue. The depth of insertion may be predetermined by the attachment of an interchangeable stop.

It is particularly important to limit the depth of injection when drugs, cells (e.g., myoblasts) or other substances are being injected into the myocardium of the heart. In such procedures, if the injector is advanced to far it may go all the way through the myocardial wall and into a chamber of the heart. If the substance is then inadvertently injected into a chamber of the heart rather than into the myocardial wall, the intended therapeutic benefit of injection into the myocardial tissue will be lost and potentially serious complications may result from the inadvertent introduction of the substance into the patient's bloodstream. One such procedure currently under development is the injection of platelet gel (PG) into an infarcted area of myocardium to improve myocardial function and/or to prevent deleterious ventricular remodeling following myocardial infarction or other injury to the myocardium. In this therapy, a platelet-containing component (e.g., platelet rich plasma (PRP)) is combined with a thrombin-containing component (e.g, a thrombin solution) immediately before, during or after injection into the myocardium at one or more location(s) within or near an infarct or other myocardial injury. The platelet-containing component (e.g., PRP) combines with the thrombin-containing component and forms a platelet gel (PG) which causes the desired therapeutic effect. Such PG is formed when components (such as fibrinogen) contained in the platelet-containing component are activated by thrombin contained in the thrombin-containing component. Autologous PRP can be obtained from the subject's own blood, thereby significantly reducing the risk of adverse reactions or infection. When autologous PRP is used as the platelet-containing component, the resultant PG is referred to as autologous platelet gel (APG). The addition of thrombin to platelet-containing plasma products such as PRP is described in detail in U.S. Pat. No. 6,444,228 and United States Patent Application Publication Nos. 2007/0014784, 2006/0041242 and 2005/209564, the disclosures of each such patent and patent application being expressly incorporated herein by reference. Since it is difficult to pass PG or APG through the lumen of a needle, it is desirable to inject the platelet-containing component and the thrombin-containing component such that they become mixed immediately prior to, during or after injection through the needle. Additionally, injecting the platelet-containing component and the thrombin-containing component separately or immediately after mixing may allow the infusate to distribute to a greater area before fully gelling into the PG or APG, thereby possibly enhancing the effect of this therapy. Multiple component injectors that are suitable for delivery of PG therapy into myocardial tissue and include optional depth stops for limiting the depth to which the injector penetrates into the myocardium are described in U.S. patent application Ser. No. 11/969,094, the disclosure of which is also expressly incorporated herein by reference.

There remains a need for the development of new devices and methods for controlling or limiting the depth to which an injector or other elongate penetrator penetrates into an organ or tissue mass.

### SUMMARY OF THE INVENTION

The present invention provides new devices and methods for controlling the depth to which a penetrator penetrates into an organ or tissue mass. As used herein the terms “angular injection” and “angular entry” indicate an injection, or the entry of a needle or penetrator, wherein the needle enters the injection site at an angle that is not perpendicular or orthogonal to the surface being penetrated by the needle. Thus, for an angular injection or angular entry to occur, the needle would not enter the injection site at a right angle to an imaginary plane that is tangent to the surface at the point of injection.

In accordance with one embodiment of the invention, there is provided a device for controlling the depth to which an elongate penetrator penetrates into an organ or other tissue mass, such device comprising; a first member comprising a penetrator shroud having a hollow bore extending therethrough and a distal end and a second member to which the penetrator is attached. The penetrator extends distally from the second member. The second member is engageable with the first member such that the penetrator extends through the bore of the penetrator shroud. The distance to which the penetrator extends beyond the distal end of the penetrator shroud is adjustable. The penetrator is then advanceable into the organ or tissue mass only until the distal end of the shroud abuts against the surface of the organ or tissue mass, thereby limiting the depth to which the penetrator can penetrate. Optionally, the distal end of the penetrator shroud may be beveled to a desired angle relative to the longitudinal axis of the penetrator to allow angular injections. The penetrator may have one or more lumens to permit aspiration or infusion or substances after it has been inserted to the desired depth of penetration.

Further in accordance with the invention, in some embodiments, the penetrator may have at least two coaxial lumens and may be used for simultaneous injection of two component substances such that the component substances become combined in situ to form a combination product. For example, a platelet rich plasma (PRP) containing component may be infused through one lumen and a thrombin containing component may be infused through another lumen such that the platelets and thrombin will combine to form platelet gel (PG) at the site of injection within the organ or tissue. In some instances, at least the PRP can be produced from a recipeint's own blood and the resulting PG will be an autologous platelet gel (APG). The site of injection may be within or near an area of impaired myocardial function and the PG or APG may have the effect of improving myocardial function and/or preventing ventricular remodeling.

Further or alternative elements, aspects, objects and advantages of the present invention will be understood by those of skill in the art upon studying of the accompanying drawings and reading of the detailed description and examples set forth below.

### BRIEF DESCRIPTION OF THE DRAWINGS

FIG. 1 is a perspective view of one embodiment of a controlled-depth injector device of the present invention.

FIG. 2 is an exploded perspective view of the device of FIG. 1.

FIG. 2A is a cross sectional view through line 2A-2A of FIG. 2.

FIG. 3 is a sagittal sectional view of a human heart with the device of FIG. 1 being used to inject PRP and Thrombin Solution to form PG at a desired location within the left ventricular wall of the heart.

FIGS. 4A through 4D show steps in a method for adjusting the needle penetration depth of the injector device of FIG. 1.

FIG. 5 is a perspective view of another embodiment of a controlled-depth injector device of the present invention.

FIG. 6 is a perspective view of yet another embodiment of a controlled-depth injector device of the present invention.

FIG. 7 is a top view of yet another embodiment of a controlled-depth injector device of the present invention.

FIG. 7A is a front perspective view of the device of FIG. 7.

FIG. 7B is a longitudinal sectional view through Line 7B-7B of FIG. 7.

FIG. 7C is an enlarged partial view of Region 7C of FIG. 7.

### DETAILED DESCRIPTION AND EXAMPLES

The following detailed description, the accompanying drawings are intended to describe some, but not necessarily all, examples or embodiments of the invention. The contents of this detailed description and accompanying drawings do not limit the scope of the invention in any way.

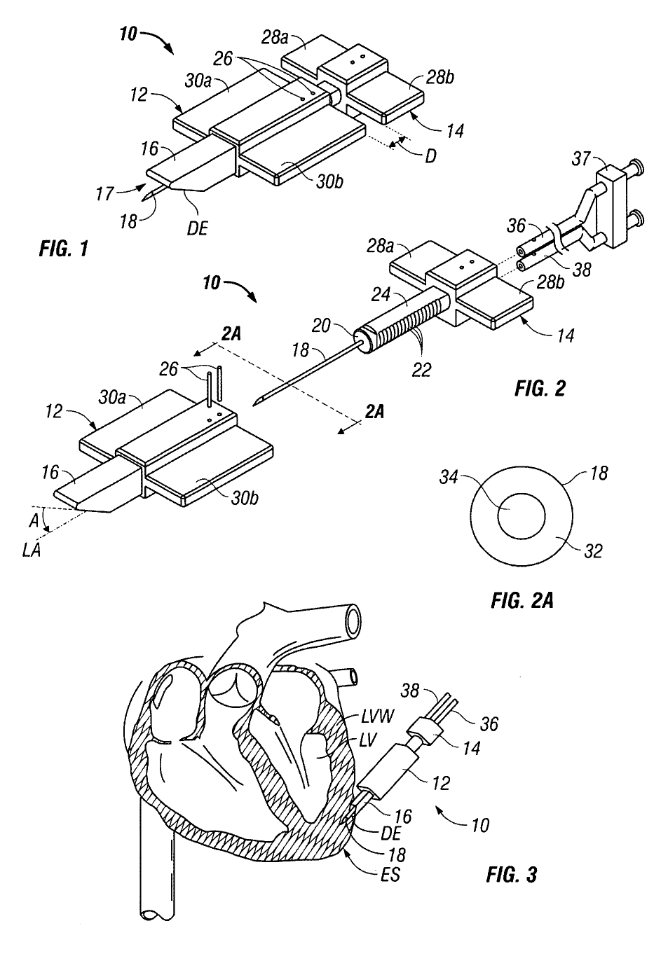

FIGS. 1 through 4B show one embodiment of a controlled-depth, multiple component injector device 10 of the present invention. This device 10 comprises a first or distal member 12 and a second or proximal member 14. A penetrator shroud 16 extends distally from the first member 12. The penetrator shroud 16 has a distal end DE. A hollow bore 17 extends through the first member 12 opening through the distal end DE of the penetrator shroud 16.

A penetrator 18 extends in the distal direction from the proximal member 14. An elongate body 20 surrounds a proximal portion of the penetrator 18. A series of vertical slots 22 are formed in one side of the elongate body 20. A flat surface 24 is formed on top of the elongate body 20. Off-center locking members 26 extend vertically through one side of the bore 17 of the first member 12. The elongate body is sized such that, when it is rotated 90 degrees such that its flat surface 24 is on the same side as the locking members 26, the flat surface 24 will pass by the locking members 26, thereby allowing the distal portion of the penetrator 18 and the elongate body 20 to be freely advanced into the bore 17 of the first member 12. Thereafter, when the proximal member 14 is rotated back to a position wherein the flat surface 24 is in top of the elongate body 20, the locking members 26 will seat within certain ones of the slots 22, thereby joining the first member 12 to the proximal member 14 such that a fixed distance D exists between the first member 12 and proximal member 14. When desired, the proximal member 14 may again be rotated 90 degrees such that the flat surface 24 of the elongate body 20 is on the same side as the locking members 26 and the proximal member may be retracted or advanced to a new position with the flat surface 24 passing by the locking members 26. After reaching the new position, the proximal member 14 may be rotated 90 degrees back to its previous rotational orientation, causing locking members 26 to seat in different ones of the slots 22. In that manner the distance D between the proximal and first members 12, 14 and the extent to which the penetrator extends beyond the distal end DE of the shroud 16 may be adjusted. Optionally, the distal end DE of the shroud 16 may be cut on an angle or bevel as shown, thereby controlling the angle or trajectory on which the penetrator 18 will advance through the tissue. In one embodiment, the angle A between the needle and the face of the distal end of the shroud is 30 degrees.

Optionally, horizontally extending wings 28a, 28b may be formed on the proximal member 14 and horizontally extending wings 30a, 30b may be formed on the first member 12 to facilitate ease of grasping and manipulating the proximal member 14 and first member 12.

The particular example of the device 10 shown in the drawings is designed for injection of 2 components. Thus, the penetrator 18 has a first lumen 32 and a second lumen 34 extending therethrough. A first component tube 38 is connectable to the proximal member 14 to infuse a first component through the first lumen 32 of the penetrator 18 and a second component tube 36 is connectable to the proximal member 14 to infuse a second component through the second lumen 34 of the penetrator 18.

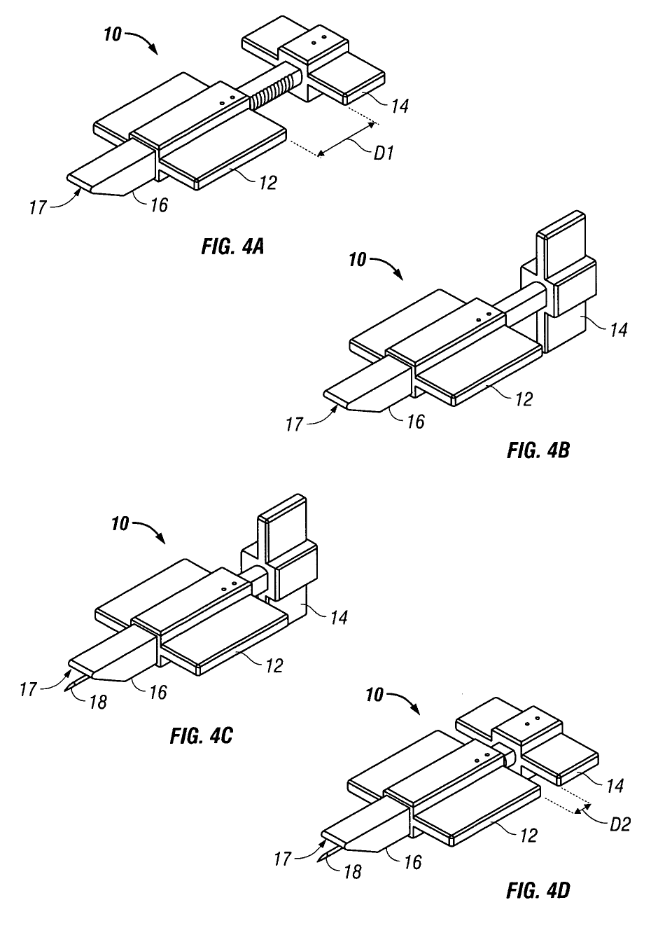

FIGS. 4A through 4D show an example of a method for preparing the device 10 to deliver an injection into an infracted zone of the left ventricular myocardial wall of a human heart. Initially, as seen in FIG. 4A, the proximal and first members 12, 14 are a spaced distance D1 apart and the penetrator 18 is within the shroud 16 such that no portion of the penetrator 18 protrudes out of the distal end DE of the shroud 16. This renders the device 10 virtually incapable if causing inadvertent needle punctures to personnel who may be handling the device 10.

Based on pre-procedure imaging studies, the wall thickness of the myocardium in the area of the infarct is known, as is the specific location of the infarcted tissue into which it is desired to deliver the injection. On that basis, the physician will determine the desired depth of injection (i.e., the distance between the epicardial surface of the heart and the center of the infarct zone). The desired depth of injection will necessarily be less than the full thickness of the myocardial wall on the intended needle trajectory, thereby avoiding the possibility of inadvertent injection of the treatment materials into the ventricle. As seen in FIG. 4B, the proximal member 14 is then rotated 90 degrees to the left, causing the locking members 16 to disengage from slots 22 and causing the flat surface 24 of the elongate body 20 to be on the from slots 22. As seen in FIG. 4C, this allows the proximal member 14 to be advanced to a position where the penetrator 18 protrudes out of and beyond the distal end of the shroud 16 by a distance that is equal to the intended depth of penetration into the myocardium.

As shown in FIG. 4D, after reaching the new position, the proximal member 14 is rotated 90 degrees back to its previous rotational orientation, causing locking members 26 to seat in different ones of the slots 22. This locks the penetrator 18 position relative to the shroud 16 so as to affect the desired penetration depth and causes the distance D2 between the first and second members 12, 14 to be reduced compared to the original distance D1 when the penetrator 18 was fully covered by the shroud.

FIG. 3 shows the manner in which the device 10, after having been prepared as described above, is used to cause a quantity of PG to be formed in situ within an infracted zone of the left ventricular wall LVW. In this example, the first supply tube 36 is connected to a source of PRP and its distal end is connected to a fitting on the proximal member 14 to inject the PRP through the inner coaxial lumen 34 of the penetrator 18. The second supply tube 38 is connected to a source of Thrombin Solution and its distal end is connected to a fitting on the proximal member 14 to inject the Thrombin Solution through the outer coaxial lumen 32 of the penetrator 18. The device 10 is held at an angle so that the angle of the beveled distal end DE of the shroud is parallel to the epicardial surface ES of the heart and the protruding portion of the penetrator 18 are aligned with the infarct zone in which it is intended to deliver the therapy. The penetrator 18 is then advanced through the epicardial surface ES and into the myocardium until the distal end DE of the shroud 16 abuts against the epicardial surface ES, thereby stopping advancement of the penetrator 18. Because the depth of penetration was pre-set, this will cause the distal end of the penetrator 18 to be within the intended infarct zone and will prevent the penetrator 18 from being inadvertently advanced too far, as could result in a misplaced injection outside of the intended infarct zone or even inadvertent entry into the left ventricle LV. Also, because the distal end of the penetrator is beveled or angled relative to the longitudinal axis LA of the penetrator, the protruding distal portion of the penetrator 18 is caused to enter the myocardium at an angle to the surface at the point of injection, as shown. The angular entry results in a longer penetrator tract, than if the penetrator 18 is inserted to the same depth by simply advancing it into the myocardium at a right angle to the epicardial surface. Having such longer penetration tract may prevent or deter unwanted backflowing of the injected substance or substances out of the penetration tract as the penetrator 18 is removed.

Thereafter, with the penetrator 18 so positioned, the PRP and thrombin solution will be simultaneously injected through the coaxial lumens 32, 34 and out of the end of the penetrator 18 causing mixing of the PRP and Thrombin Solution and resultant in situ formation of a quantity of PG within the infarct zone as described in detail in the above-incorporated U.S. patent application Ser. No. 11/969,094. In at least some embodiments, the PRP and thrombin solution may be delivered at a ratio of about 10 parts PRP to 1 part thrombin solution. In embodiments where flexible supply tubes 36, 38 or other flexible member(s) is/are attached to the device 10, allow an operator to insert the penetrator into a heart for an angular injection while allowing the device 10 to be sufficiently free to undergo some movement along with natural myocardial motions of the beating heart. Because the device is not rigidly connected to any syringes or other infusion apparatus, the infusion apparatus does not undergo any movement caused by the motion of the heart. This provides a clinician with better control of the injection because the infusion apparatus can be manipulated separately from the injection device.

After the therapeutic substances have been injected, the device 10 may be removed and the procedure shown in FIGS. 4A through 4B may be performed in reverse to return the penetrator 18 to a fully shielded position within the shroud 16, thereby avoiding inadvertent penetrator trauma to personnel who subsequently handle or dispose of the device 10.

Those of skill in the art will appreciate that potential uses of this embodiment of the device 10 having the penetrator 18 with coaxial lumens 32, 34, are not limited to delivery of PG therapy to the myocardium but may be used to deliver virtually any two-component therapy or diagnostic material to any organ, tissue mass, cavity, lumen or other target location. Other examples of two-component materials that may be delivered using this device 10 include, but are not limited to, two component tissue adhesives and sealants (e.g., Tisseel VH™ Fibrin Sealant, available commercially from Baxter Healthcare Corporation, Deerfield, Ill.) and tissue bulking agents, fillers or polymeric materials (e.g., hydrogels) that may be fomred or expanded in situ for various therapeutic or cosmetic applications such as tissue bulking, filling or expanding and various prodrug+activator combinations.

Also, the devices of the present invention need not be used only for two component delivery. Instead, the penetrator 18 may have a single lumen for delivery of a single material or any number of additional lumens (3 or more) for delivery of multiple component therapies having more than two components.

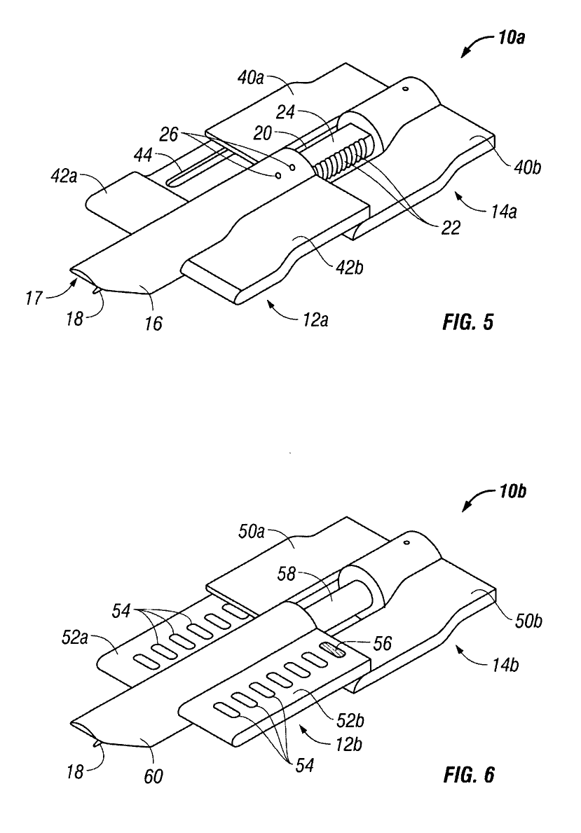

Also, the particular configuration and construction of the device 10 shown in FIGS. 1 through 4D is only one example and various alternative configurations or constructions may be employed. One alternative configuration and construction is seen in the device 10a of FIG. 5. This device 10a differs from the device 10 of FIGS. 1-4D in that it has contoured, overlapping wing members 40a, 40b, 42a, 42b, as shown. Additionally, this device 10a includes an anti-rotation lock that deters inadvertent or accidental rotation of the second or proximal member 14a relative to the first or first member 12a, as such unintended rotation could cause the locking members 26 to be unseated from grooves 22 allowing unwanted proximal or distal movement of the proximal member 14a relative to the first member 12a. Specifically, as the proximal member 14a is advanced toward the first member 12a, a distal portion of proximal wing member 40a will lap over distal wing member 42a and a distal portion of proximal wing member 40b will lap under distal wing member 42b. The anti-rotation lock in the particular embodiment shown comprises the combination of a groove 44 formed in the upper surface of distal wing member 42a and a projection (not shown) on the underside of proximal wing member 40a which will snap fit into groove 44, thereby preventing unintended or accidental rotation of the proximal member 14a relative to the first member 12a.

Another alternative configuration and construction is seen in the device 10b of FIG. 6. This device 10b comprises a first or distal member 12b and a second or proximal member 14b. The first member 12b comprises a penetrator shroud 60 which extends distally, as shown. The penetrator shroud 60 has a hollow bore that extends longitudinally therethrough. Optionally, the distal end surface of the penetrator shroud 60 may be beveled or angled as described above. First and second wing members 52a, 52b extends laterally on either side of the penetrator shroud 60 and a row of spaced-apart depressions or locking apertures 54 are formed in each wing member 52a, 52b. A smooth-surfaced, non-threaded projection 58 (e.g., a cylindrical boss) extends in the distal direction from the center of the second member 14b and the penetrator 18 (described above) extends in the distal direction from the non-threaded projection 58. This non-threaded projection 58 is received within the hollow bore of the penetrator shroud 60 and freely slides back and forth within such bore. An upwardly curved wing member 50a is formed on one side of the second member 14b and a downwardly curved wing member 50b is formed on the other side of the second member 14b, as shown. A first locking projection (not seen in FIG. 6) protrudes downwardly from the undersurface of first wing member 50a and a second locking projection 56 protrudes upwardly from the upper surface of the second wing member 50b. These locking projections 56 are sized to insert within and frictionally engage (e.g., “snap-fit”) respective ones of the locking apertures 54 formed in the wings 52a, 52b of the first member 12b. While the first member 12b is held in a substantially fixed rotational orientation, the second member 14b may be rotated in the counterclockwise direction sufficiently to cause the locking projections 56 to be pulled out of and disengaged from locking apertures 54. The non-threaded projection 58 may then be advanced in the distal direction through the bore of the penetrator shroud 60 until a desired length of the penetrator 18 protrudes out of and beyond the distal end of the penetrator shroud 60. When such desired position has been reached, the second member 50b may then be counter-rotated in the clockwise direction to cause the locking projections 56 to be received within and to frictionally engage (e.g., “snap fit”) adjacent ones of the locking apertures 54. This will hold the first and second members 12b, 14b is fixed positions relative to one another with the desired length of penetrator 18 protruding out of and beyond the distal end of the penetrator shroud 60, thereby controlling the depth of penetration into an organ or tissue as described in detail above.

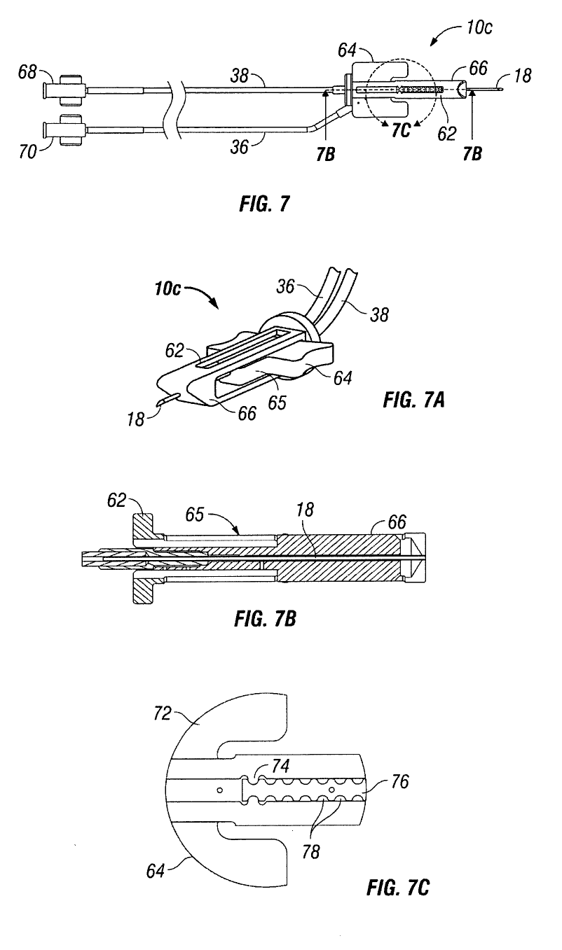

Another alternative configuration and construction is seen in the device 10c of FIGS. 7 through 7C. This device comprises a first member 62 and a second member 64. The first member 62 comprises a penetrator shroud 66 having open side slots 65 and an open distal end. Wing members 72 extend laterally from either side of the second member 64. The second member 64 is positioned within the first member 62 such that its wing members 72 protrude outwardly through the side slots 65. A penetrator 18 of the type described above is affixed to and extends in the distal direction from the second member 64. A linear series of teeth or projections 74 are formed on the second member 64 and the first member 62 comprises a rack 76 having a corresponding series of notches or depressions 78. The projections 74 are biased to seat within the adjacent depressions 78. When it is desired to adjust the depth to which the penetrator 18 will penetrate, the first member 62 is held in a relatively fixed longitudinal position and the second member 64 is advanced in the distal direction and/or or retracted in the proximal direction with sufficient force to overcome the bias of the projections 74 causing projections 74 to slide over depressions 78 until a desired length of the penetrator 18 extends out of and beyond the distal end of the penetrator shroud. When such position is reached, the projections will once-again seat within adjacent ones of the depressions 78, thereby substantially holding the first and second members 62, 64 in fixed positions relative to one another with the desired length of penetrator 18 protruding out of and beyond the distal end of the penetrator shroud 66, thereby controlling the depth of penetration into an organ or tissue as described in detail above. In this example, the penetrator 18 has dual lumens and flexible tubes 36, 38 are attached to the device 10c. Syringes or other infusion apparatus may be attached to Luer connectors 68, 70 on the proximal ends of the flexible tubes 36, 38 to inject desired quantities of substances through the lumens of the penetrator 18 as described above.

It is to be further appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless to do so would render the embodiment or example unsuitable for its intended use. Also, where the steps of a method or process are described, listed or claimed in a particular order, such steps may be performed in any other order unless to do so would render the embodiment or example not novel, obvious to a person of ordinary skill in the relevant art or unsuitable for its intended use. All reasonable additions, deletions, modifications and alterations are to be considered equivalents of the described examples and embodiments and are to be included within the scope of the following claims.

What is claimed is:

1. A device for controlling the depth to which an elongate penetrator penetrates into an organ or other tissue mass, said device comprising:
a first member comprising a penetrator shroud having a hollow bore extending therethrough and a distal end; and
a second member, to which the penetrator is attached, the penetrator extending distally from the second member, said second member being engageable with the first member such that the penetrator extends through the bore of the penetrator shroud;
the distance to which the penetrator extends beyond the distal end of the penetrator shroud being adjustable.

2. A device according to claim 1 wherein the penetrator has at least one lumen so as to be useable for aspiration of matter or delivery of substance(s).

3. A device according to claim 2 wherein the penetrator has at least two lumens so as to be useable for the simultaneous injection of two substances.

4. A device according to claim 2 where one lumen of the penetrator is connected to a first flexible supply tube.

5. A device according to claim 4 wherein a second lumen of the penetrator is connected to a second flexible supply tube.

6. A device according to claim 5 wherein substance delivered through one flexible supply tube remains isolated from substance delivered through the second supply tube until both substances have exited the penetrator.

7. A device according to claim 1 further comprising a flexible member attached to the device, said flexible member allowing the penetrator to be inserted into a heart or other anatomical structure, while allowing the device to undergo some movement concurrent with movement of a beating heat or other moving anatomical structure into which the penetrator has been inserted.

8. A device according to claim 1 where the distal end of the shroud is at an angle relative to the penetrator axis to allow for angular injections relative to the organ or tissue mass when the distal end of the shroud is flush against said organ or tissue mass.

9. A device according to claim 1 wherein at least one wing is formed on the second member.

10. A device according to claim 9 wherein first and second wing members are formed at diametrically opposite locations on the second member.

11. A device according to claim 1 wherein at least one wing is formed on the first member.

12. A device according to claim 11 wherein first and second wing members are formed at diametrically opposite locations on the first member.

13. A device according to claim 1 wherein the second member may be rotated relative to the first member between a first rotational position whereby the penetrator is held in a fixed longitudinal position relative to the shroud and a second rotational position whereby the penetrator is allowed to be longitudinally advanced or retracted relative to the shroud.

14. A device according to claim 13 further comprising an anti-rotation lock that locks the second member in the first rotational position thereby deterring inadvertent longitudinal movement of the penetrator relative to the shroud.

15. A device according to claim 14 wherein the first and second members have wings that overlap one another when the second member is in the first rotational position and wherein the anti-rotation lock comprises a groove on one of said wings and a projection on the other of said wings, the projection being snap-fittable into the groove to frictionally hold the second member in the first rotational position relative to the first member.

16. A method for advancing an elongate penetrator to a desired depth within an organ or issue mass, said method comprising the steps of:
(A) providing a device that comprises a first member and a second member, the penetrator being attached to and extending distally from the second member, the first member having a penetrator shroud that has a distal end and a hollow bore extending therethrough, the second member being engageable with the first member such that the penetrator extends through the hollow bore and the distance to which the penetrator protrudes out of and beyond the distal end of the penetrator shroud being adjustable;
(B) determining the desired depth of penetration into the organ or tissue mass;
(C) adjusting the device such that the penetrator extends beyond the distal end of the shroud bay a distance that is substantially equal to the desired depth of penetration; and
(D) advancing the penetrator into the organ or tissue mass until the distal end of the shroud abuts against the organ or tissue mass.

17. A method according to claim 16 wherein the penetrator has a lumen and wherein the method further comprises the step of aspirating matter into or through the penetrator lumen.

18. A method according to claim 16 wherein the penetrator has a lumen and wherein the method further comprises the step of injecting a substance through the penetrator lumen.

19. A method according to claim 18 wherein the penetrator has a plurality of lumens and wherein a plurality of substances are injected through the penetrator.

20. A method according to claim 19 wherein a first component comprising platelets is injected through one penetrator lumen and a second component comprising thrombin is injected through another penetrator lumen, causing the platelets and thrombin to combine to form platelet gel (PG) or autologous platelet gel (APG).

21. A method according to claim 20 wherein the first component comprises Platelet Rich Plasma (PRP) and the second component comprises a thrombin-containing solution.

22. A system according to claim 21 wherein the injectors are sized such that the PRP and thrombin solution become combined at a ratio of about 10 parts PRP to 1 part thrombin solution.

23. A method according to claim 18 wherein the penetrator is advanced into the myocardium until the distal end of the shroud abuts against the epicardial surface of the heart.

24. A method according to claim 23 wherein the penetrator is advanced to a location within or near an area of impaired myocardial function and wherein the substance that is injected has a therapeutic effect within said area of impaired myocardial function.

25. A method according to claim 23 wherein the adjustment made in Step D substantially prevents the penetrator from being advanced into a chamber of the heart.

26. A method according to claim 16 wherein the device provided in Step A further comprises a lock which holds the penetrator in a substantially fixed longitudinal position relative to the shroud and wherein the method further comprises using said lock to hold the penetrator in a substantially fixed longitudinal position relative to the shroud after making the adjustment in Step C.

27. A method according to claim 16 wherein Step B comprises performing an imaging study and determining the desired depth of penetration from the imaging study.

28. A method according to claim 23 performed on a beating heart, wherein the device further comprises at least one flexible member attached to the device and wherein said at least one flexible member allows the penetrator to be inserted into a heart, while at least one substance is injected through the penetrator and the device undergoes some movement along with the beating of the heart.

29. A method according to claim 28 wherein said at least one flexible member comprises at least one flexible substance supply tube through which at least one substance is delivered through the penetrator.
